(19) United States
(12) Patent Application Publication
(10) Pub. No.: US 2019/0356771 A1
(43) Pub. Date: Nov. 21, 2019

(54) Smart Notification System

(71) Applicant: QUALCOMM Incorporated, San Diego, CA (US)

(72) Inventors: Balamurugan Selvarajan, Chennai (IN); Sham Muthayyan, Chennai (IN); Jaiganesh Narayanan, Chennai (IN)

(21) Appl. No.: 15/982,010

(22) Filed: May 17, 2018

(51) Int. Cl. H04M 1/725; A61B 5/00; G04G 13/02

(52) U.S. Cl. CPC H04M 1/72569; G04G 13/02; A61B 5/4809; H04M 1/72566

(57) ABSTRACT

A method and apparatus for smart notification system. The apparatus comprises a contextual sensor for sensing a status related to a first activity. A notification component outputs a notification related to the first activity when the status meets a first condition. A processor is coupled to the contextual sensor and the notification component, and initiates disabling or re-enabling of the notification component.

### BACKGROUND

Wireless communication technologies have seen explosive growth over the past years. This growth has been fueled by better communications and hardware, larger networks, and more reliable protocols. As a result, wireless service provides are now able to offer users with unprecedented levels of access to information, resources, functions, and applications. Therefore, mobile terminals, such as mobile phones, are relied upon for many functions other than just for a phone communication. With increasing applications pre-installed and available from App stores, a mobile phone can be used to watch video, listen to music, play games, as well as perform monetary transactions

One common application used on a mobile phone is an alarm related to wake-up or a calendar event. Consider a case where a user wakes up before the alarm wakes the user but forgets to disable or turn off the alarm. Even if the user is already awake, the alarm would then still go off and disturb the user. Therefore, there is need for a smart alarm system that can automatically disable the alarm if it is no longer needed

### SUMMARY

Various embodiments include methods of sensing contextually the surrounding environment and controlling a notification component based on the sensed status.

Some embodiments include a contextual sensor configured to sense a status related to a first activity; a notification component configured to output a notification related to the first activity when the status meets a first condition; and a processor coupled to the contextual sensor and the notification component, wherein the processor initiates disabling or re-enabling of the notification component. The notification component may be either one or a combination of an alarm unit, a display unit, an audio unit, or a motion unit. Also, the contextual sensor may comprise either one or a combination of an audio sensor, a visual sensor, a motion sensor, or a temporal sensor.

In some embodiments, the smart notification may be implemented in a wake-up notification such as an alarm device. The contextual sensing may be done either by one or a combination of monitoring for audible sounds; monitoring for visual status; sensing motion; or monitoring time.

Apparatus for implementing the smart notification may include means for setting a notification time for notifying a user at a notification time to perform a first activity; means for receiving an indication of the first activity being performed before the notification time, means for determining at the notification time whether the activity has been performed; means generating a notification if the activity has not yet been performed at the notification time; or means for disabling the notification if the activity has been performed at the notification time. The means may include a non-transitory processor-readable storage medium having stored thereon processor-executable instructions configured to cause a processor of an apparatus to perform operations. The means may include a processor configured to set a notification time for notifying a user at the notification time to perform a first activity; wherein the processor is further configured to receive an indication of the first activity being performed before the notification time, and determine at the notification time whether the activity has been performed; a notification component, coupled to the processor, configured to generate a notification if the activity has not yet been performed at the notification time; or disable the notification if the activity has been performed at the notification time.

### BRIEF DESCRIPTION OF THE DRAWINGS

The accompanying drawings, which are incorporated herein and constitute part of this specification, illustrate example embodiments of the claims, and together with the general description given above and the detailed description given below, serve to explain the features of the claims.

FIG. 1 is an example device that can implement the inventive concepts.

FIG. 2 is an example flow diagram of a method that can implement the inventive concepts.

FIG. 3 is an example system implementing the inventive concepts.

### DETAILED DESCRIPTION

Various embodiments will be described in detail with reference to the accompanying drawings. Whenever possible, the same reference numbers will be used throughout the drawings to refer to the same or like parts. References made to particular examples and implementations are for illustrative purposes and are not intended to limit the scope of the claims. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of mobile devices will now be presented with reference to various apparatus and methods. These apparatus and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

Also, the terms “communications device” and “computing device” are used interchangeably herein to refer to any one or all of cellular telephones, smart phones, smart watches or wearables, personal or mobile multi-media players, personal data assistants (PDAs), laptop computers, tablet computers, smart books, palm-top computers, wireless electronic mail receivers, multimedia Internet enabled cellular telephones, wireless gaming controllers, and similar personal electronic devices that include at least a processor. Various embodiments would also include a memory and/or storage as well as circuitry for establishing wireless communications pathways and transmitting/receiving data via wireless communications pathways.

Communications devices may use a variety of interface technologies, such as wired interface technologies (e.g., Universal Serial Bus (USB) connections, etc.) and/or air interface technologies (also known as radio access technologies) (e.g., Third Generation (3G), Fourth Generation (4G), Long Term Evolution (LTE), Edge, Bluetooth, Wi-Fi, satellite, etc.). Communications devices may establish connections to a network, such as the Internet, via more than one of these interface technologies at the same time (e.g., simultaneously). For example, a mobile communications device may establish an LTE network connection to the Internet via a cellular tower or a base station at the same time that the mobile communications device may establish a wireless local area network (WLAN) network connection (e.g., a Wi-Fi network connection) to an Internet connected Wi-Fi access point.

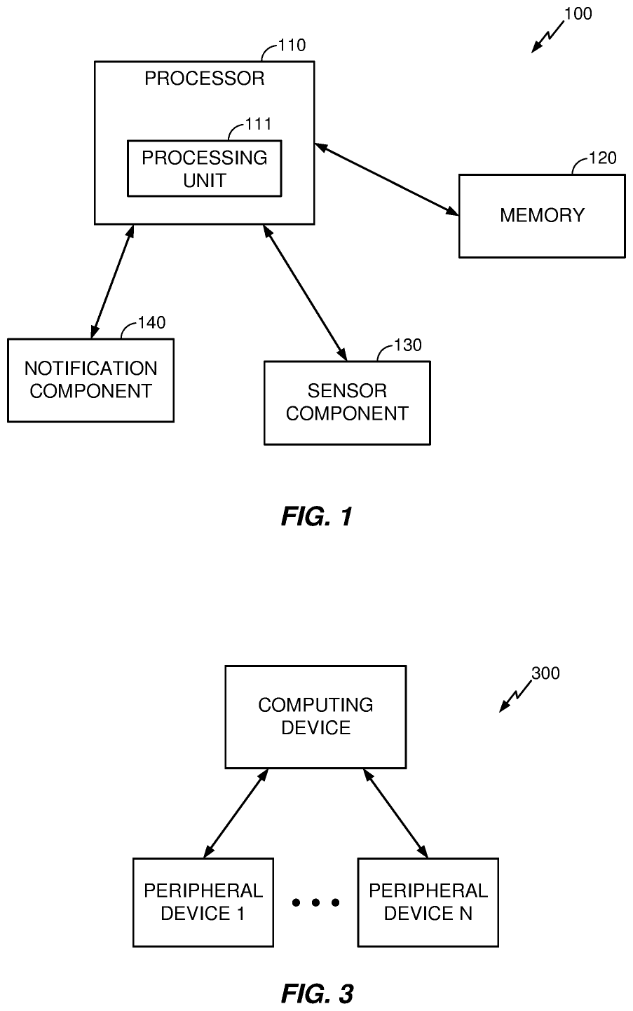

FIG. 1 is a schematic diagram illustrating components of a computing device 100 that may be configured to enable and perform a smart notification. The computing device 100 may include a processor 110, a memory 120, a sensor component 130 and a notification component 140. Various embodiments of the computing device 100 may include other circuits and/or components (not shown) such as various modems, a clock, a display component, various user interfaces, and transceivers configured to support the transmission and reception of messages.

Processor 110 may be dedicated hardware specifically adapted to perform various operations of the computing device 100, including, but not limited to, executing an operating system and/or various instances of one or more programs (i.e., processes). In some embodiments, the processor 110 may be or include a programmable processing unit 111 that may be programmed with processor-executable instructions to perform the various operations of the computing device 100. In some embodiments, the processor 110 may be a programmable microprocessor, microcomputer or multiple processor chip or chips that can be configured by software instructions to perform the various operations of the computing device 100. In some embodiments, the processor 110 may be a combination of dedicated hardware and a programmable processing unit 111.

In some embodiments, the memory 120 may store processor-executable instructions. In some embodiments, the memory 120 may be volatile memory, non-volatile memory (e.g., flash memory), or a combination thereof. In some embodiments, the memory 120 may include internal memory included in the processor 110, memory external to the processor 110, or a combination thereof. In some embodiments, the memory 120 may include volatile memory, such as a random access memory (RAM), in which an operating system and various instances of one or more programs (i.e., processes) may be executed by the processor 110.

While the various components of the computing device 100 are illustrated in FIG. 1 as separate components, some or all of the components may be integrated together in a single device such as a mobile device or phone, or a module, such as a system-on-chip module.

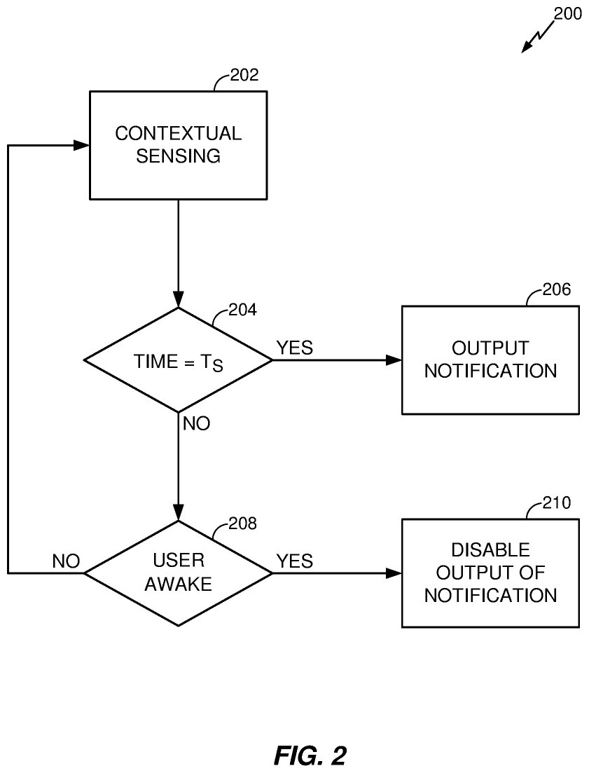

FIG. 2 illustrates a method 200 of performing smart notifications in a computing device. For ease of reference, the processor or processes performing the operations within the computing device are referred to generally herein as a “processor.”

In block 202, computing device contextually senses the status associated with a first activity which may be various events and/or application settings. In various embodiment, the first activity may be a wake-up notification that a user would set on the computing device. For example, the user may activate a notification to emit an audible sound or alarm at 8 am in the morning. In this example, the computing device may track time and if the status indicates that the time has reached 8 am, the computing device would emit an alarm. The time reaching tₛ, 8 am in this example, would be a first condition in block 204. However, if the first condition has not been met, i.e., the time has not reached 8 am, the computing device, in block 206, determines if the sensed status meets a second condition. In this example, if the status indicates that the user is already awake and/or active before 8 am, then the computing device in block 212 may disable the notification.

In some embodiments, after the computing device senses that the user is awake, it may be that the user has awoken prematurely and may go back to asleep. For example, the user may get up to use the restroom or get a cup of water. Therefore, in various embodiments, the computing device may further determine if there is still a predetermined time period t₁ remaining before the first condition is met. The predetermined time period t₁ may be set by user, may be preset at manufacture or may be preset by other third parties such as a carrier. If for example, t₁ has been set to 30 minutes, then the computing device would not disable the output of the notification unless the time has reached 7:30 am. The computing device would continue to contextually sense the status of the first condition.

In alternative embodiments, the computing device would disable the notification if the status indicates that the user is awake. Thereafter though, if the sensed status indicates that the user is no longer awake, the computing device may re-enable or re-activate the notification.

While the method has been described with reference to an audible sound or alarm, the notification may be activated in various ways. Examples include but is not limited to a display of a visual output and/or generation of motion. For a visual display, the computing device would include a display screen (not shown) which may, for example, flash lights or output messages and/or graphical image. For generation of motion, the computing device may include a small motor (also not shown) that would cause vibration of the computing device.

In various embodiments, a sensor component would contextually sense a status associated with the first activity. The sensing may be accomplished in one or a combination of many ways, including but not limited to, tracking time, detecting motion, monitoring for visual status, monitoring for audible sounds and receiving at least one input signal from one or more peripheral devices. FIG. 3 illustrates an environment 300 by which the sensor component of a computing device may receive signal(s) from peripheral device(s).

FIG. 3 shows a computing device in communication with peripheral devices 1-n. The communication may be wireless, wired, or a combination of both. The peripheral device may be a variety of devices capable of sensing contextually the environment, processing and transmitting signals to the computing device. It may be a very smart device such as another computing device or may be a simple device implementing a small processor and a transmitter. Generally, the peripheral device would be somewhat near the computing device. Examples of a peripheral device includes a television, an alarm clock, a camera, smart lights, a refrigerator, other appliances, and wearable devices.

In various embodiments, a processor would determine whether the first condition and second condition have been met as well as enable, disable and/or re-enable the notification component. Also, in some embodiments, the notification component may be external to the computing device in which case, the computing device would send at least one output signal to one or more peripheral devices such as illustrated in FIG. 3. These peripheral devices would include a receiver to receive signals from the computing device. The signal would indicate disabling and/or re-enabling of the notification output. One example of an external or peripheral device would be a speaker or multimedia device connected with the computing device. Also, while a wake-up notification was used as the first activity, other events and/or applications may implement the smart notification.

The foregoing method descriptions and the process flow diagrams are provided merely as illustrative examples, and are not intended to require or imply that the operations of the various embodiments must be performed in the order presented. As will be appreciated by one of skill in the art, the operations in the foregoing embodiments may be performed in any order. Words such as “therefore,” “then,” “next,” etc. are not intended to limit the order of the operations; these words are simply used to guide the reader through the description of the methods. Further, any reference to claim elements in the singular, for example, using the articles “a,” “an,” or “the” is not to be construed as limiting the element to the singular.

As used in this application, the terms “component,” module,” “system,” and the like are intended to include a computer-related entity, such as, but not limited to hardware, firmware, a combination of hardware and software, software, or software in execution, which are configured to perform particular operations or functions. For example, a component may be, but is not limited to, a process running on a processor, a processor, an object, an executable, at thread of execution, a program, and/or a computer. By way of illustration, both an application running on a computing device and the computing device may be referred to as a component. One or more components may reside within a process and/or thread of execution, and a component may be localized on one processor or core and/or distributed between two or more processor or cores. In addition, these components may execute from various non-transitory computer readable media having various instructions and/or data structures stored thereon. Components may communicate by way of local and/or remote process, functions or procedure calls, electronic signals, data packets, memory read/writes, and other known network, computer, processor, and/or process related communication methodologies.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks modules, circuits, ad operations have been described above generally in terms of their functionality. Whether such functionality is implemented as hardware or software depends upon the particular application and design constrains imposed on the overall system. Skilled artisans may implement the described functionality in varying ways for each particular application, but such implementation decisions should not be interpreted as causing a departure from the scope of the claims.

The preceding description of the disclosed embodiments is provided to enable any person skilled in the art to make or use the claims. Various modifications to these embodiments will be readily apparent to those skilled in the art, and the generic principles defined herein may be applied to other embodiments without departing from the scope of the claims. Thus, the present disclosure is not intended to be limited to the embodiment shown herein but is to be accorded the widest cope consistent with the following claims and principles and novel features disclosed herein.

What is claimed is:

1. An apparatus comprising:
a contextual sensor configured to sense a status related to a first activity;
a notification component configured to output a notification related to the first activity when the status meets a first condition; and
a processor coupled to the contextual sensor and the notification component, wherein the processor initiates disabling or re-enabling of the notification component.

2. The apparatus of claim 1, wherein the notification component may be either one or a combination of an alarm unit, a display unit, an audio unit, or a motion unit.

3. The apparatus of claim 1, wherein the contextual sensor may comprise either one or a combination of an audio sensor, a visual sensor, a motion sensor, or a temporal sensor.

4. The apparatus of claim 1, wherein the apparatus is a mobile phone.

5. A method comprising:
sensing contextually a status related to a first activity;
outputting a notification related to the first activity when the status meets a first condition; and
disabling the notification when the status meets a second condition.

6. The method of claim 5, wherein the first activity is a wake-up notification, the first condition is when the status indicates that current time is a wake-up time, and the second condition is when the status indicates that a user is awake.

7. The method of claim 6, further comprising:
re-activating the wake-up notification when the status indicates that the user is no longer awake.

8. The method of claim 7, wherein re-activating the wake-up notification further comprises:
re-activating the wake-up notification when the status indicates that the user is no longer awake and time remaining until the wake-up time is greater than a first period of time.

9. The method of claim 6, wherein disabling a notification further comprises:
disabling the notification when the status indicates that the user is awake and time remaining until the wake-up time is less than a second period of time.

10. The method of claim 5, wherein outputting the notification comprises one or a combination of:
emitting an audible sound;
displaying a visual output; or
generating a motion.

11. The method of claim 5, further comprising:
disabling the notification related to the first activity when the status meets a second condition.

12. The method of claim 5, wherein the sensing contextually comprises either one or a combination of:
receiving at least one input signal from one or more peripheral devices;
monitoring for audible sounds;
monitoring for visual status;
sensing motion; or
monitoring time.

13. The method of claim 5, wherein disabling the notification further comprises sending at least one output signal to one or more peripheral devices; wherein the output signal indicates disabling of the notification.

14. An apparatus comprising:
a processor configured to set a notification time for notifying a user at the notification time to perform a first activity; wherein the processor is further configured to receive an indication of the first activity being performed before the notification time, and determine at the notification time whether the activity has been performed; and
a notification component, coupled to the processor, configured to generate a notification if the activity has not yet been performed at the notification time; or disable the notification if the activity has been performed at the notification time.

15. A method comprising:
setting a notification time for notifying a user at a notification time to perform a first activity;
receiving an indication of the first activity being performed before the notification time,
determining at the notification time whether the activity has been performed;
generating a notification if the activity has not yet been performed at the notification time; or
disabling the notification if the activity has been performed at the notification time.

16. An apparatus comprising:
means for setting a notification time for notifying a user at a notification time to perform a first activity;
means for receiving an indication of the first activity being performed before the notification time,
means for determining at the notification time whether the activity has been performed;
means generating a notification if the activity has not yet been performed at the notification time; or
means for disabling the notification if the activity has been performed at the notification time.

17. A non-transitory processor-readable storage medium having stored thereon processor-executable instructions configured to cause a processor of an apparatus to perform operations comprising:
setting a notification time for notifying a user at a notification time to perform a first activity;
receiving an indication of the first activity being performed before the notification time,
determining at the notification time whether the activity has been performed;
generating a notification if the activity has not yet been performed at the notification time; or
disabling the notification if the activity has been performed at the notification time.
